(19) United States
(12) Patent Application Publication
(10) Pub. No.: US 2006/0280759 A1
(43) Pub. Date: Dec. 14, 2006

(54) Live and subunit vaccines

(76) Inventors: Richard William Titball, Salisbury (GB); Stephen Lloyd Michell, Salisbury (GB); Petra Claire Farquhar Oyston, Salisbury (GB); Anna-Lena Forslund, Umea (SE); Kerstin Kuoppa, Umea (SE); Kerstin Svensson, Umea (SE); Emelie Salonmonsson, Umea (SE); Anders Johansson, Umea (SE); Laila Noppa, Umea (SE); Mona Bystrom, Umea (SE); Elisabet Frithz-Lindsten, Umea (SE); Mats Forsman, Umea (SE); Ake Forsberg, Umea (SE)

Correspondence Address: JOHN S. PRATT, ESQ;KILPATRICK STOCKTON, LLP, 1100 PEACHTREE STREET, ATLANTA, GA, 30309, US

(21) Appl. No.: 11/217,228

(22) Filed: Sep. 1, 2005

(30) Foreign Application Priority Data
Jun. 8, 2005 (GB) 0511722.1

(51) Int. Cl. A61K 39/02

(52) U.S. Cl. 424/234.1; 424/236.1

(57) ABSTRACT

Immunogenic compositions containing Francisella components or modified live strains of the Francisella species and their use in preventing or treating disease such as tularemia.

### CROSS REFERENCE TO RELATED APPLICATION

This application claims priority to Great Britain Patent Application No. 0511722.1 filed on Jun. 8, 2005, the contents of which are incorporated herein by reference.

### BACKGROUND

The present invention relates to new immunogenic compositions, containing components of Francisella bacteria or containing live strains of the Francisella species, and use of the compositions in preventing or treating disease.

Francisella tularensis is the causative agent of tularemia and is a member of the bacterial family Francisellaceae. The three species within the genus Francisella are F. tularensis, Francisella novicida and Francisella philomiragia. A 16S ribosomal DNA sequence analysis has placed the genus Francisella as a member of the γ subclass of the proteobacteria. The F. tularensis species was originally divided into two biotypes, A and B, but recently four recognizable biotypes have been proposed. F. tularensis subspecies tularensis, previously known as Type A or subspecies nearatica, is recognized as the most virulent. It is responsible for human cases of tularemia in North America and Europe and causes severe disease in mammals, especially rabbits. F. tularensis subspecies palaearctica, also known as holartica or Type B, is found in Europe, Asia and North America and is less virulent in humans than F. tularensis subspecies tularensis. F. tularensis subspecies media asiatica has been isolated from central Asia and subspecies palaearctica japonica is found only in Japan.

F. tularensis subspecies philomiragia was originally known as the “Philomiragia” bacterium before being renamed Yersinia philomiragia. It was finally placed in the Francisella genus on the basis of biochemical tests and cellular fatty acid analysis. Although F. tularensis subspecies novicida and F. tularensis subspecies philomiragia are considered pathogenic to humans they pose only a small risk.

F. novicida was classified into the genus Pasteurella in 1955, but then reclassified in 1959 into the genus Francisella. It was initially considered a separate species to F. tularensis, however recently it has been proposed that it should be designated F. tularensis subspecies novicida because of the similarities between the two species. Both of these designations are utilized herein.

At the genetic level this similarity to F. tularensis is greater than 99% and the two species are chemically and antigenically very similar, demonstrating strong serological cross-reactivity. However, it has been discovered by applicants that F. novicida can be differentiated from F. tularensis on the basis of less fastidious growth requirements and the ability to produce acid from sucrose. F. novicida is fully virulent in the mouse model with a LD₅₀ of 1.76 cfu, but has reduced virulence in humans when compared to F tularensis.

Human cases of tularemia usually result from bites from vectors such as biting flies, ticks and mosquitoes that have recently fed on an infected animal. However, there have been reported cases of infections caused by contact with dead animals, infectious aerosols, and ingestion of contaminated food and water. Hunters, veterinarians, walkers and farmers are at the greatest risk of contracting tularemia because they are likely to come into contact with infected animals. The incidence of tularemia in humans is usually low, but an increase in the number of cases is observed when there is an epidemic in the local animal reservoir.

In the 1940s, attempts were made to produce killed vaccines against tularemia consisting of whole killed cells or cell extracts. However these attempts failed to give protection against challenge with fully virulent strains. Subsequent efforts have concentrated on the production of a live vaccine. Live attenuated strains were developed in the former Soviet Union by repeatedly passaging the bacterium on media containing antiserum. Several strains were suitably attenuated for use as a vaccine and were used as such, either alone or in a mixed culture vaccine.

In 1956, a mixture of strains of Francisella tularensis were transferred from the former Soviet Union to the United States. From these, a suitably attenuated strain was isolated and tested for safety and efficacy and was designated F. tularensis live vaccine strain (LVS). The vaccine was delivered via the scarification route using a dose of 0.06 ml and was followed by yearly boosters. Retrospective studies on the efficacy of the LVS vaccine based on laboratory acquired infections have shown that it affords good but not complete protection against typhoidal tularemia leading to a dramatic decrease in cases.

Although the incidence of the ulceroglandular form of tularemia was not decreased, a reduction in the severity of the clinical symptoms was observed. Studies using F. tularensis LVS have shown that protection is correlated with cell-mediated immunity. Protein antigens on the surface of the bacterium induce the cell-mediated response. However, a large number of antigens appear to be important because there is no bias in the response towards one particular antigen. It has been found that the cytokines interleukin-1 and interferon-γ are important in providing resistance to infection. The humoral response induced by carbohydrate antigens on the bacterium also has a role in protection but can only protect against challenge by strains with reduced virulence.

The LVS vaccine is not registered and was only used to vaccinate at-risk personnel under special license, however this licenses has now been withdrawn. The main reason for this is that the genetic changes responsible for the attenuating phenotype are not understood at the molecular level. Therefore, the possibility exists that the vaccine strain could revert back to the fully virulent form. A new vaccine is therefore required.

### SUMMARY OF THE INVENTION

Immunogenic compositions containing components of Francisella bacteria or modified live strains of the Francisella species are provided. The immunogenic compositions are useful in the preparation of vaccines or Francisella subunit vaccines for preventing or treating disease, such as tularemia.

Therefore, an immunogenic composition is described herein that contains either a strain of Francisella species, wherein the strain has inactivated pili, in combination with a pharmaceutically acceptable carrier, or the immunogenic composition contains a pilin antigen in combination with a pharmaceutically acceptable carrier. Prophylactic and therapeutic uses of the immunogenic compositions are described herein.

### BRIEF DESCRIPTION OF THE DRAWINGS

FIG. 1 Three genes, FTT0890, FTT0889, and FTT0888, with specific features common for the type IV pili building block protein (Clemens, D. L., Lee, B. Y. & Horwitz, M. A. (2004) Infect. Immun. 72, 3204-3217) located in a region of direct repeats. (A-B) Schematic alignment of the three genes in different Francisella strains and the unidirectional repeat mediated deletion mechanism. The symbol Δ represents the deleted sequence. Direct repeats (DR1 and DR2) flanking the deletion and the resulting composite repeat are depicted as hatched bars. Open bars represent open reading frames that were truncated by the deletion. (C) The FSC354 nucleotide sequence with flanking repeats, DR1 and DR2 underlined, before and after (composite repeat) the deletion of the intervening sequence.

FIG. 2 Presence and transcription of pilin genes in wild type and complemented strains of F. tularensis. Localization of different primer pairs (A-E) based on the SCHU S4 sequence is indicated. (A) PCR on chromosomal DNA demonstrates that the pilin genes (FTT0890, 0889, and 0888) are present in SCHU S4 (FSC237), FSC352, FSC354, and FSC507 (the cis-complemented FSC354/pil⁺), but a 500 bp deletion even has occurred in FSC354. (B) RT-PCR on mRNA using primer pairs A-E. Controls consisting of reaction mixtures in which reverse transcriptase was all omitted were all negative (data not shown).

FIG. 3 Expression of pilin protein in different pathogenic strains of F. tularensis identified by a F. tularensis pilin antisera. (A-B) Denatured total lysate from wild type strains and the two pilin gene complemented strains of F. tularensis, FSC498 and FSC507 (Table 2). (C) Cell surface protein from wild type and pilin complemented strains of F. tularensis separated on SDS-PAGE under non-denaturing conditions.

FIG. 4 F. tularensis pilin gene was expressed by the heterologous host P. aeruginosa. Whole cell lysates of wild type F. tularensis strains, SCHU S4 (FSC237) and FSC074, and the pilA deletion strain PAKpilA⁻ of P. aeruginosa complemented with a synthetic F. tularensis pilin gene in trans. Prior to immunodetection with F. tularensis pilin antiserum, the complemented PAK strain was grown under IPTG inducing conditions.

FIG. 5 The pilin gene is expressed in FSC508 (FSC354 complemented in trans with pil⁺ flag). (A) Non-denatured total lysates were separated on SDS-PAGE and proteins were identified by an α-Flag antibody and pili antiserum. (B) The strains FSC508 and FSC352 were analysed by fluorescence microscopy using the α-Flag antibody.

FIG. 6 pAL 10 harboring the pilin gene is stable in vivo. Eight days after infection bacteria were recovered from the spleens. Total lysates from four harvested colonies were denatured, separated on SDS-PAGE and analyzed by immunodetection with pilin antibodies. Lanes: g, FSC352; h, FSC498 (pilA trans-complemented FSC354) before infection; i-1, FSC498 8 days after infection (in vivo).

FIG. 7 The diagram illustrates a typical pattern of the kinetics in the infection study. Mice in groups of three or five were infected subcutaneously with different strains. Note the correlation of the kinetics between FSC352 and FSC507 when mice are infected with less than 10 bacteria. Notable is also the similarity of the infection pattern between strain FSC354 and FSC498, where the infection dose of FSC498 is 10 times lower.

### DETAILED DESCRIPTION OF THE INVENTION

Immunogenic compositions containing modified live strains of the Francisella species in combination with a pharmaceutically acceptable carrier are described herein. Immunogenic compositions containing components of Francisella bacteria in combination with a pharmaceutically acceptable carrier are also described herein. The immunogenic compositions are useful in the preparation of vaccines or Francisella subunit vaccines for preventing or treating disease, such as tularemia. The modified live strains of the Francisella species provided in the immunogenic compositions contain inactivated pili. The components of Francisella bacteria provided in the immunogenic compositions include one or more pilin antigens. Prophylactic and therapeutic uses of the immunogenic compositions are also described.

Francisella and Pilins

In order to colonize host cells, a bacterium must have the facility to adhere to the host surface. The best understood mechanism of adherence is attachment via rod-shaped protein structures called “pili”, or “fimbriae”. Conventionally, “fimbriae” is the correct term for these surface structures and “pili” is supposed to be reserved for the longer, more flexible rodlike structures involved in conjugation. However, as used herein, the terms “pili” and “pilus” refer to any rod-shaped bacterial surface structure containing pilin.

Many bacteria have pili; Escherichia coli, for instance, attaches to membranes of the urinary or genital tracts of humans by means of their pili. In cholera, long filamentous pili make an important contribution to colonization of the intestinal mucosa. The attachment results from specific binding between the pili and specific receptors, probably glycoproteins, on the host cell surface. The term “adhesin” has been given to the proteins in pili that attach the pili to the receptors on the host cell surface.

Francisella Type IV Pili

The recently published genome sequence of the highly virulent type A strain SCHU S4 of F. tularensis (Svensson, K., Larsson, P., Johansson, D., Byström, M., Forsman, M. & Johansson, A. (2005) J. Bacteiol., accepted for publication in the June issue.) revealed the presence of various gene clusters thought to be involved in virulence. Of particular interest was the fact that the genome was found to encode all the genes required for expressing functional type IV pili.

Type IV pili are complex surface organelles present on many human bacterial pathogens such as Neisseria., Pseudomonas aeruginosa, and Vibrio cholerae. They are thought to be essential for establishment of infection because, like other pili, they mediate critical interactions with host cells. They have also been found to be involved in DNA uptake, biofilm formation and twitching motility. Type IV pili proteins are divided into two subclasses, IVA and IVB, based on the presence of conserved motifs.

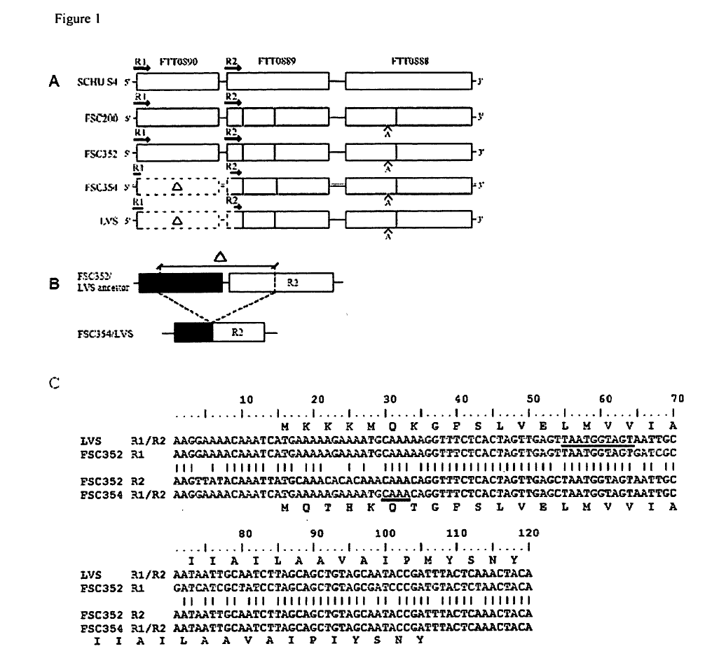

When the genome sequence of strain SCHU S4 was analyzed for genes and operons encoding proteins predicted to have a function in assembly and secretion of type IV pili, one region was found that encoded three genes (FTT0888, FTT0889 and FTT0890), herein referred to as the pilin FTT0888 gene, pilin FTT0889 gene and pilin FTT0890 gene respectively) with typical signatures for type IV pilins and one of the genes (FTT0890) was found to be flanked by two 120 bp repeats. In most type B strains isolated from outbreaks of tularemia in Sweden, it was only FTT0890 that was intact and the two downstream pilin-like genes appeared to be non-functional due to nonsense mutations (FIG. 1A).

The gene sequence for the pilin FTT0888 gene is as follows.

(SEQUENCE ID NO:1)

ATGATAACTGTAAAATCAAAAAAAGGTTTTTCACTTACTGAGTTACTTG
TTGTTATAGCTATTATAGGCATTTTGTCGATACTAGTTATCCCAGCGTA
TTCAAACTTATGTCACTAGAACAAGATTGACAGAAGCCTTGACAGTTTT
AGATGGTTATAAAAAAGATGTCCAATCTTATTTTATTGCGCATGGTGTC
TCAACCCAAGAACAGTTAGAAAATTATGATGTCACAGATCATGCTGATG
TTGGTAGGGATACTACTAGCGTAATGAGTAATATAGTAGGTCATAACGG
TAGAATAGTTGGTGTAAGCACGATCAAAGGCACAACTTACCAAATAGCT
CTAACTCCAAGGATAGAGGGTGGTACATTCAACTGGACATGCTCTATTT
CAATAGTTGAACAAGACGCTAGCTTTGATAATAGTTATAATGGTCAAGT
CTTCTTTGGATTTATTGATAATAATAGTGGCTCATATGCCGCTCCAAGC
AGTAGTATGCTACCAAGAGGTTGTAATGCTACAGATAGCAACCAAAATA
CTGATTTCGAAGCATATAGATCAGAAAGACAGACACTAGATAACAGAGC
TTATGAATAG

The gene sequence for the pilin FTT0889 gene is as follows.

(SEQUENCE ID NO:2)

ATGCAAACACACAAACAAACAGGTTTCTCACTAGTTGAGCTAATGGTAGT
AATTGCAATAATTGCAATCTTAGCAGCTGTAGCAATACCGATTTACTCAA
GCTACAAAGAGCGTGCTGCAATTATCGAATCTATGAACATAATTGGTAAT
GTCAAAGCTAGTATCCAAAATGATATGAATAATAATCTAGATATCTCCCA
GCAAACTTATGACACCCCTACTGGAGTCACTGTAACAGGTAGTACGTCAG
GAGCTACTATTGATATAAATCTAAGCCAAACTTCACCACAACACTTTACC
AACGATAATGATATTATCAGACTTAGTGGTGTAGTTGTTAGTGGTAGCAC
TTTCCAATGGACTTGCTCACATAATGTCAACGCCTCAACATTAACAGCTA
GTAATGTCCCACACACTTGCTCAAGTACTTTTAGTGCTTAG

The gene sequence for the pilin FTT0890 gene is as follows.

(SEQUENCE ID NO:3)

ATGAAAAAGAAAATGCAAAAAGGTTCTCACTAGTTGAGTTAATGGTAGTG
ATCGCGATCATCGCTATCCTAGCAGCTGTAGCGATCCGGATGTACTCTAA
CTACACTACACGTGCTCAGTTAGGCTCTGATCTATCTGCTCTAGGTGGTG
GTAAAGCTACAGTAGCTGAAAGAATAGCTAACAACAATGGTGATGCATCT
CAAGTTACAATTCTTCAAGCTAATGCCGCTGCAAATGGTCTTCCAAGTGG
TGCTTCAGTTGCTGCTGGTACTATTAGTTATCCATCAACAGTATCTGGTG
CAACAATTCAATTAGCTCCTACAGTAAGTTCCGGTGCTATTACTTGGACT
TGTAATATTTCAGGTGTATCAGCATCTCAAGTACCATGTAACTGTAATGC
TATCTAA

The polypeptide sequence for pilin FTT0888 is as follows.

(SEQUENCE ID NO:4)

MITVKSKKGFSLTELLVVIAIIGILSILVIPAYSNYVTRTRLTEALTVLD
GYKKDVQSYFIAHGVSTQEQLENYDVTDHADVGSDTTSVMSNIVGHNGRI
VGVSTIKGTTYQIALTPRIEGGTFNWTCSISIVEQDASFDNSYNGQVFFG
FIDNNSGSYAAPSSSMLPRGCNATDSNQNTDFEAYRSERQTLDNRAYE

The polypeptide sequence for pilin FTT0889 is as follows.

(SEQUENCE ID NO:5)

MQTHKQTGFSLVELMVVIAIIAILAAVAIPIYSSYKERAAIIESMNIIGN
VKASIQNDINNNLDISQQTYDTPTGVTVTGSTSGATIDINLSQTSPQHFT
NDNDIIRLSGVVVSGSTFQWTCSHNVNASTLTASNVPHTCSSTFSA

The polypeptide sequence for pilin FTT0890 is as follows.

(SEQUENCE ID NO:6)

MKKKMQKGFSLVELMVVIAIIAILAAVAIPMYSNYTTRAQLGSDLSALGG
AKATVAERIANNNGDASQVTILQANAAANGLPSGASVAAGTISYPSTVSG
ATIQLAPTVSSGAITWTCNISGVSASQVPSNCNAI

The pilin FTT0888, FTT0889 and FTT0890 genes all encode type IV pili fiber building block proteins (see GenBank Accession number AJ749949). Comprehensive studies of the Francisella genome sequence revealed that more than 70 regions in the genome are flanked by long direct repeats of DNA and, in some strains, genes surrounded by the flanking regions have been found to have deletions, a result of a mechanism involving homologous recombination between the direct repeats.

It has been discovered herein that the presence of functional type IV pili is absolutely critical for the virulence of F. tularensis infected via the subcutaneous route in mice and that virulence can be restored by restoring the functional type IV pili genes in complementation studies. This discovery therefore shows that functional type IV pili are essential for the ability of Francisella to infect via peripheral routes.

Type IV pili have been extensively studied in gram-negative pathogens, which are mostly extracellular during infection. The finding that type IV pili are required in the virulence of an intracellular pathogen like Francisella is therefore surprising and the role of such pilin in an intracellular pathogen is not immediately apparent.

The fact that the virulence defect is only apparent via peripheral infection routes indicates that the type IV pili may be the key to the spread of the pathogen from the initial site of infection. This implies that the pilus mediates critical interactions at the site of infection after, for instance, an insect bite (i.e. vector-borne transmission), and possibly also at later stages to allow the pathogen to proliferate and cause a systemic infection.

Compositions Containing Modified Live Strains of Francisella

In a first aspect of the immunogenic composition provided herein, the composition contains a modified live strain of Francisella species in combination with a pharmaceutically acceptable carrier, wherein the strain is modified so that it has inactivated pili. By “a strain” having “inactivated pili” is meant any strain having any gene involved in the biosynthesis, export or secretion of type IV pili onto the bacterial surface, being down-regulated. Such down-regulation could include reduced transcription of the gene, reduced translation of the gene or the mis-folding of the resulting polypeptide.

By an “immunogenic composition”, as used herein, is meant a composition that is capable of generating an immune response in a host organism. An “immune response” is defined as the reaction of the body to a foreign or potentially dangerous substance. Such a response can be an innate immune response or an adaptive immune response, or both. In particular, the immune response is a protective immune response which protects the host organism from subsequent challenge with a pathogen.

Immunogenic compositions include antigen-containing compositions. An “antigen” means any substance that the body regards as foreign and that elicits an immune response. An antigen includes a molecule that is capable of inducing the formation of an antibody. An antigen may include a polypeptide.

Preferably, the modified strain has fewer than 100, 90, 80, 70, 60, 50, 40, 30, 20, 10, 9, 8, 7, 6, 5, 4, 3, 2, or 1 pili exhibited on the cell surface of a typical cell of that strain. By a “typical cell” is meant the mean average number of pili exhibited by 10 randomly individual bacteria cells prepared for transmission electron microscopy as described in Gil, H., Benach, J. L. & Thanassi, D. G. (2004) Infect. Immun. 72, 3042-3047. Most preferably, no pili are exhibited on the cell surface.

It has been discovered that the pilin FTT0890 gene in LVS has been deleted by a recombination event mediated by the flanking direct repeats. However, other type IV pilin genes appear to be functional for LVS, and Gil et al. (Gil, H., Benach, J. L. & Thanassi, D. G. (2004) Infect. Immun. 72, 3042-3047) have published data suggesting that the vaccine strain LVS expresses surface pili that resemble type IV pili. Expression of such type IV pili on the surface of LVS has not been verified, and sheared material from LVS was found to contain very few pili-like structures. It is possible that LVS expresses some pili, albeit a reduced number compared to wild-type virulent strains, and that these pili have different properties compared to the wild-type strains. This is supported by the preliminary findings that it is not possible to cis-complement LVS by introduction of the pilin FTT0890 gene. However, in a trans-complemented LVS strain, the FTT0890 pilin gene was found to be transcribed even though protein expression levels were very low. This suggests that additional genes, important for export/assembly of type IV pili, are either not functional or not expressed in LVS. What is described herein for the first time, is that in the specific case of the pilin FTT0890 gene, the loss of the gene is irreversible and there is no risk for reversion of this particular gene in LVS. Even though this may be reassuring for using LVS as a vaccine strain, LVS is still not a preferred vaccine because, as explained above, the full genetic changes responsible for the attenuated phenotype is not understood and, as was found to be the case, the loss of the FTT0890 gene may be only one of the causes of attenuation. Preferably, therefore, the modified live strain of the Francisella species in the immunogenic composition described herein is not LVS. In particular, the modified live strain of the composition contains at least one gene which is downregulated compared to LVS, provided that this is preferably other than the FTT0890 gene.

The Francisella strain of the modified live strain of the Francisella species in the immunogenic composition described herein may be from a type A or type B Francisella species. Preferably, the strain is a strain of Francisella tularensis. More preferably, the strain is a strain of Francisella tularensis subspecies tularensis or a strain of Francisella tularensis subspecies novicida.

In a first embodiment of the modified live strain of the Francisella species in the immunogenic composition described herein, the immunogenic composition contains a strain of Francisella having a deletion or mutation in a type IV pilin gene. A “type IV pilin gene” as used herein, means any gene that is related to the biosynthesis, secretion or export of type IV pili to the Francisella cell surface.

Genetic Analysis of Type IV Pilin Gene Cluster

The genome sequence of the virulent strain SCHU S4 identified all currently known genes necessary for type IV pili biosynthesis (Larsson, P., Oyston, P. C., Chain, P., Chu, M. C., Duffield, M., Fuxelius, H. H., Garcia, E., Halltorp, G., Johansson, D., Isherwood, K. E. et al. (2005) Nat Genet. 37, 153-159) and these genes are distributed on different operons. Table 1 summarizes the type IV pili genes found to be present in the SCHU S4 genome.

TABLE 1

| GenBank | | | | |
|---|---|---|---|---|
| Accession | | | Gene | |
| Number | GI number | gene ID | name | Description |
| YP_169162 | 56707266 | 3190704 | pilT | Type IV pili nucleotide-binding protein |
| YP_169283 | 56707387 | 3191339 | | Type IV pili fiber building block protein |
| YP_169699 | 56707803 | 3191481 | pilD | Type IV pili leader peptidase and methylase |
| YP_169863 | 56707967 | 3191727 | | Type IV pili fiber building block protein |
| YP_169885 | 56707989 | 3191703 | | Type IV pili fiber building block protein |
| YP_169886 | 56707990 | 3191704 | | Type IV pili fiber building block protein |
| YP_169887 | 56707991 | 3191700 | | Type IV pili fiber building block protein |
| YP_169902 | 56708006 | 3191692 | | Type IV pili glycosylation protein |
| YP_170038 | 56708142 | 3192032 | | Type IV pili lipoprotein |
| YP_170105 | 56708209 | 3191922 | pilB | Type IV pili nucleotide binding protein, ABC |
| | | | | transporter, ATP-binding protein |
| YP_170106 | 56708210 | 3192189 | pilC | Type IV pili polytopic inner membrane protein |
| YP_170123 | 56708227 | 3191514 | pilQ | Type IV pilin multimeric outer membrane protein |
| YP_170124 | 56708228 | 3191399 | | Type IV pili lipoprotein |
| YP_170125 | 56708229 | 3191034 | | Type IV pili glycosylation protein |
| YP_170126 | 56708230 | 3191614 | | Type IV pili associated protein |

Preferably, the deletion or mutation in the type IV pilin gene occurs in any one of a group of gene sequences including, but not limited to, gene ID 3190704, ID 3191339, ID 3191481, ID 3191727, ID 3191703, ID 3191704, ID 3191700, ID 3191692, ID 3192032, ID 3191922, ID 3192189, ID 3191514, ID 3191399, ID 3191034, or ID 3191614. More preferably, there is a deletion or mutation in a further type IV pilin gene. Even more preferably, the deletion or mutation in the further type IV pilin gene is in any one of the group of gene sequences including, but not limited to, gene ID 3190704, ID 3191339, ID 3191481, ID 3191727, ID 3191703, ID 3191704, ID 3191700, ID 3191692, ID 3192032, ID 3191922, ID 3192189, ID 3191514, ID 3191399, ID 3191034, or ID 3191614. The gene sequences are available in the GenBank database at the National Center for Biotechnology Information (NCBI) at the National Library of Medicine. (Bethesda, Md., U.S.A.) (See also the Entrez website at ncbi.nlm.nih.gov/entrez/ or ncbi.nlm.nih.gov/entrez/query.fcgi?db=gene) More preferably still, the deletion or mutation in the type IV pilin gene occurs in one or more of the type IV pilin FTT0888 gene, FTT0889 gene or FTT0890 gene sequences.

In a second embodiment of the modified live strain of the Francisella species in the immunogenic composition described herein, the type IV pilin gene is partially deleted or mutated so that it encodes an antigenic analog of a naturally occurring Francisella polypeptide.

Preferably, the naturally occurring Francisella polypeptide is a polypeptide from the SCHU S4 strain of F. tularensis. More preferably, the naturally occurring Francisella polypeptide includes, but is not limited to, a sequence selected from the group GI56707266, GI56707387, GI56707803, GI56707967, GI56707989, GI56707990, GI56707991, GI56708006, GI56708142, GI56708209, GI56708210, GI56708227, GI56708228, GI56708229 and GI56708230.

A “naturally occurring Francisella polypeptide”, as used here in, refers to any polypeptide with an amino acid sequence that occurs within a naturally occurring Francisella strain. “Naturally occurring” as used herein, means anything that is to be found in nature, without having been subjected to genetic manipulation by molecular biology techniques. The original SCHU S4 strain is an example of a “naturally occurring” strain. Molecular biology techniques are well known to the skilled person and include techniques such as cloning, polymerase chain reaction, restriction cutting, etc. Such methods can be found in any laboratory manual (see for example, MOLECULAR CLONING by J. Sambrook, E. F. Fritsch, T. Maniatis, 2nd ed., Cold Spring Harbor, N.Y., Cold Spring Harbor Laboratory Press, 1989).

An “antigenic analog” as used with respect to a polypeptide, describes any antigen that is capable of stimulating an immune response which is protective or therapeutic against Francisella infection in a dose-dependent manner and shares more than 60% identity or similarity with a polypeptide sequence from the genome of a naturally occurring Francisella strain, along the length of the antigen sequence. Preferably, the length of the antigen sequence corresponds to the polypeptide encoded by a Francisella gene. Preferably the antigen sequence shares more than 30, 35, 40, 45, 50, 55, 60, 65, 70, 75, 80, 85, 90, or 95% identity or similarity with the corresponding Francisella genome sequence along the length of the antigen sequence. More preferably, the antigen sequence shares more than 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity or similarity with the corresponding Francisella genome sequence along the length of the antigen sequence.

The “antigenic analog” may be a polypeptide that is homologous or analogous to the Francisella polypeptide. The two terms “homologous” and “analogous” as used herein, are used interchangeably. Two polypeptides are said to be “homologous” or “analogous”, if the sequence of one of the polypeptides has a high enough degree of identity or similarity to the sequence of the other polypeptide, that is, they share more than 30, 35, 40, 45, 50, 55, 60, 65, 70, 75, 80, 85, 90 or 95% similarity or identity along the length of the antigen sequence. More preferably, the two sequences share more than 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity or similarity along the length of the antigen sequence. “Identity”, when referring to a polypeptide, indicates that at any particular position in the aligned sequences the amino acid residue is identical between the sequences. “Similarity”, when referring to a polypeptide, indicates that, at any particular position in the aligned sequences, the amino acid residue is of a similar type between the sequences. For example, amino acid residues can be grouped by their side chains. Glycine, alanine, valine, leucine and isoleucine all have aliphatic side chains and amino acids in this group may be regarded as similar. Proline, although a cyclic amino acid, shares many properties with the aliphatic amino acids and may also be regarded as being grouped with the other aliphatic amino acids. Another group is the hydroxyl or sulphur containing side chain amino acids. These are serine, cysteine, threonine and methionine. Phenylalanine, tyrosine and tryptophan are grouped together as the aromatic amino acids. Histidine, lysine and arginine are the basic amino acids. Aspartic acid and glutamic acid are the acidic amino acids and asparagine and glutamine are their respective amides. Also included in these groups are modified amino acids (i.e. non-naturally occurring amino acids) that have side-chains that share similar properties with the naturally occurring amino acids. Members of a particular group can be regarded as being “similar”. Swapping one amino acid from a group with another amino acid from the same group is often termed a conservative substitution.

The definition of a “homologous” or “analogous” polypeptide may also include a polypeptide that has had one or more amino acids deleted or inserted into the sequence, as long as the overall identity or similarity is 30, 35, 40, 45, 50, 55, 60, 65, 70, 75, 80, 85, 90 or 95% along the length of the antigen sequence. The amino acids that are inserted or substituted may be non-conservative amino acid changes as long as the overall identity or similarity falls within the given percentages. Homologous or analogous polypeptides may include further natural biological variants.

Degrees of identity and similarity can be readily calculated using known computer programs (see COMPUTATIONAL MOLECULAR BIOLOGY, Lesk, A. M., ed., Oxford University Press, New York, 1988; BIOCOMPUTING. INFORMATICS AND GENOME PROJECTS, Smith, D. W., ed., Academic Press, New York, 1993; COMPUTER ANALYSIS OF SEQUENCE DATA, Part 1, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). For example, simple sequence comparisons can be done on web-sites such as the NCBI website: http://www.ncbi.nlm.nih.gov/BLAST/ (version 2.2.10).

“Antigenic analogs” also includes fragments of the naturally occurring Francisella polypeptide, provided that the fragments share at least one of the antigenic determinants occurring in the naturally occurring Francisella polypeptide. Two polypeptides share the same antigenic determinant if they both bind to a particular antibody with similar binding affinities. Techniques for measuring the binding affinities of proteins with proteins are well known in the art and may include filter binding studies, ELISAs or chromatography. Binding affinities are regarded as being similar when they are statistically significant.

In a preferred embodiment, the fragments of the naturally occurring Francisella polypeptide, are fragments that share at least one of the antigenic determinants occurring in a sequence such as, but not limited to, the group GI56707266, GI56707387, GI56707803, GI56707967, GI56707989, GI56707990, GI56707991, GI56708006, GI56708142, GI56708209, GI56708210, GI56708227, GI56708228, GI56708229 and GI56708230.

In a particularly preferred embodiment of the first aspect of the invention, the Francisella strain present in the immunogenic composition is a live strain wherein a gene has been inactivated, and which is able to produce a protective immune response in an animal. “Live strain”, as used herein, refers to a whole organism, which may or may not have been manipulated to become attenuated. This is in contrast to immunogenic compositions containing individually expressed heterologous Francisella polypeptides, e.g. when the immunogenic composition is used as a subunit vaccine. A “protective immune response”, as used herein means that the immunogenic composition generates an adaptive immune response in the host organism.

Compositions Containing Components of Francisella

In a second aspect of the immunogenic composition provided herein, the composition contains a component of Francisella bacteria, preferably a type IV pilin antigen, or an antigenic analog thereof, in combination with a pharmaceutically acceptable carrier. An “antigen” is as defined above. A “type IV pilin antigen”, as used herein, describes any type IV pilin related molecule that is an antigen to a host organism. A “type IV pilin related molecule” is any molecule that is involved in the biosynthesis, secretion or export of type IV pili to the cell surface of a microorganism. Preferably, the type IV pilin antigen is a polypeptide. Where the “antigenic analog” is a polypeptide, the definition is as described above. Alternatively, the type IV pilin antigen may be another type of molecule, e.g. a polynucleotide.

In one embodiment, the pilin antigen is a Francisella antigen, or an antigenic analog thereof. A “Francisella antigen”, as used herein, describes any type of molecule that originates from a Francisella strain and is an antigen to the host organism. A “molecule that originates from a Francisella strain”, as used herein means that the molecule was originally identified as being present in a Francisella strain, for example, it may have been part of the genome, or it may have been a polypeptide that is expressed by a gene present in the Francisella genome. A “Francisella antigen” may include a polynucleotide containing a sequence that is identical or similar to a sequence in the Francisella genome, or may include a polypeptide that is identical or similar to known or predicted Francisella polypeptides. The Francisella antigen may be heterologously produced, e.g. by way of a vector.

Preferably, the Francisella antigen originates from a Francisella type A or type B species, or is an antigenic analog thereof. More preferably, the Francisella antigen originates from a Francisella tularensis strain, e.g. a Francisella tularensis subspecies tularensis strain or Francisella tularensis subspecies novicida strain, or is an antigenic analog thereof. Even more preferably, the pilin antigen originates from the SCHU S4 strain of F. tularensis. Still more preferably, the pilin antigen is a polypeptide containing a sequence from GI56707266, GI56707387, GI56707803, GI56707967, GI56707989, GI56707990, GI56707991, GI56708006, GI56708142, GI56708209, GI56708210, GI56708227, GI56708228, GI56708229 or GI56708230, or is an antigenic analog thereof. Most preferably, the pilin antigen is a polypeptide containing a sequence selected from the type IV pilin FTT0888, FTT0889 or FTT0890 sequences, or is an antigenic analog thereof. The antigenic analogs may include fragments, as described previously.

In a second embodiment of the immunogenic composition containing a component of Francisella bacteria, the immunogenic composition includes a polynucleotide capable of encoding a polypeptide pilin antigen. Preferably, the polynucleotide encodes a polypeptide containing a sequence, including, but not limited to, GI56707266, GI56707387, GI56707803, GI56707967, GI56707989, GI56707990, GI56707991, GI56708006, GI56708142, GI56708209, GI56708210, GI56708227, GI56708228, GI56708229 or GI56708230, or encodes an antigenic analog of the polypeptide. More preferably, the polynucleotide of the second embodiment, contains a sequence selected from the gene ID 3190704, ID 3191339, ID 3191481, ID 3191727, ID 3191703, ID 3191704, ID 3191700, ID 3191692, ID 3192032, ID 3191922, ID 3192189, ID 3191514, ID 3191399, ID 3191034 or ID 3191614. Most preferably, the polynucleotide contains the gene sequence from the type IV pilin FTT0888 gene, FTT0889 gene or FTT0890 gene sequences.

A “polynucleotide” as used herein, is any molecule containing more than one nucleic acid. The polynucleotide of the invention may comprise nucleic acids that are deoxyribonucleic acid (DNA), ribonucleic acid (RNA) or modified nucleic acids, or a combination thereof. The polynucleotide may include cDNA, synthetic DNA, or genomic DNA. The RNA may be mRNA or synthetic RNA.

“Modified nucleic acids” include analogs of DNA and RNA such as those containing modified backbones and peptide nucleic acids (PNA). The term “PNA”, as used herein, refers to a polynucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8: 53-63).

The polynucleotide described herein may be double-stranded or single-stranded. Single-stranded DNA may be the coding strand, also known as the sense strand, or it may be the non-coding strand, also referred to as the anti-sense strand.

The polynucleotides provided herein may be obtained by cloning, by chemical synthetic techniques, or by a combination thereof. Molecular cloning techniques are well known in the art (see for example, MOLECULAR CLONING by J. Sambrook, E. F. Fritsch, T. Maniatis, 2nd ed., Cold Spring Harbor, N.Y., Cold Spring Harbor Laboratory Press, 1989). DNA molecules may generally be synthesized in vitro by processes such as polymerase chain reaction (PCR). RNA molecules may generally be generated by the in vitro or in vivo transcription of DNA sequences. Variant or modified polynucleotides of the present invention may also be synthesized using random fragmentation and PCR reassembly of gene fragments. Site-directed mutagenesis may be used to insert new restriction sites, alter glycosylation patterns, change codon preference, produce splice variants and introduce mutations.

A polynucleotide “capable of encoding a pilin antigen”, as used herein, means that the polynucleotide can be a sequence that is bound by a start and a stop codon and can be continuously translated into a polypeptide that is a pilin antigen or is an antigenic analog thereof. Alternatively, the polynucleotide can be processed by living organisms, organelles or enzymes to produce an open reading frame sequence that encodes an antigen of the first aspect of the invention. For example, polynucleotide sequences that incorporate introns that are excisable by eukaryotic organisms, organelles or enzymes, polynucleotide sequences that include alternative splicing sequences, leader sequences or secretory sequences, additional sequences which encode additional amino acids, such as those which provide additional functionalities are included in the definition. Also included are those polynucleotide sequences that encode a single polypeptide containing more than one protein that is subsequently cleaved into discrete proteins or polypeptides. For example, retroviruses have their surface and transmembrane proteins initially synthesized as a single polypeptide and is then modified and cleaved by a cell-encoded protease during transport to the surface of the cell. Polynucleotides encoding more than one protein in a single polypeptide wherein the polypeptide is capable of being proteolytically cleaved to produce a pilin antigen of the second aspect of the invention, are also included.

A polynucleotide containing a sequence “capable of encoding a pilin antigen”, means that, although the polynucleotide of the second aspect of the invention may be identical to a particular polynucleotide sequence disclosed herein (e.g. pilin FTT0890 gene sequence), the polynucleotides provided herein may also include an variant on such sequences based on the redundant nature of the genetic code. In other words, because more than one codon translates into one amino acid, (for example the amino acid histidine can be encoded by the polynucleotide sequence CAU or CAC), then a particular polypeptide can be the result of a translation of different polynucleotides.

The variants may be naturally-occurring variants such as allelic variants or non-naturally occurring variants. The variants may differ from the polynucleotide sequences disclosed in the present invention by nucleotide deletions, insertion or substitutions. The substitutions, deletions or insertions may involve one or more nucleotides. Alterations may produce conservative or non-conservative amino acid substitutions, deletions or insertions. Non-naturally occurring variants of the polynucleotide may be made by mutagenesis techniques, including those applied to nucleic acid molecules, cells or organisms. In all cases, the polynucleotide variants must satisfy encode a polypeptide that is a type IV pilin antigen.

Additionally, the polynucleotide of the second embodiment of the second aspect of the invention may encode an antigenic analog of the polypeptide type IV pilin antigen. In such a case, the antigenic analog must encode or comprise an antigenic determinant.

In one embodiment of the second aspect of the invention, the polynucleotide of the second embodiment, contains at least “n” consecutive nucleotides from the polynucleotide sequences disclosed herein, where n is 10 or more, more preferably 12, 14, 16, 18, 20, 22, 24, 26, 28, 30, 35, 40, 45, 50, 55, 60 or more, or is a polynucleotide that is complementary thereof.

In a preferred embodiment, the polynucleotide of the second embodiment is at least 70% identical to any one of the sequences from gene sequences containing gene ID 3190704, ID 3191339, ID 3191481, ID 3191727, ID 3191703, ID 3191704, ID 3191700, ID 3191692, ID 3192032, ID 3191922, ID 3192189, ID 3191514, ID 3191399, ID 3191034, or ID 3191614, over the length of gene, or contains the complementary sequence thereof. More preferably, the polynucleotide contains a region that is at least 90%, more preferably still, at least 95%, 98% or 99% identical to any one of the sequences including, but not limited to, gene ID 3190704, ID 3191339, ID 3191481, ID 3191727, ID 3191703, ID 3191704, ID 3191700, ID 3191692, ID 3192032, ID 3191922, ID 3192189, ID 3191514, ID 3191399, ID 3191034, or ID 3191614, over the length of gene, or contains the complementary sequence thereof.

In a particularly preferred embodiment, the polynucleotide of the second embodiment is at least 70% identical to any one of the sequences selected from type IV pilin FTT0888, FTT0889 or FTT0890 gene sequences over the length of gene, or contains the complementary sequence thereof. More preferably, the polynucleotide contains a region that is at least 90%, more preferably still, at least 95%, 98% or 99% identical to any one of the sequences selected from type IV pilin FTT0888, FTT0889 or FTT0890 gene sequences over the length of gene, or contains the complementary sequence thereof.

In a third aspect of the immunogenic composition provided herein, the composition of the first or second aspect further includes an adjuvant. The term “adjuvant”, as used herein, means a substance that enhances the immune response of a host organism to an antigen. Adjuvants are added to antigens when a strong immunogenic response is required, such as when the antigen is being delivered as a vaccine to an animal.

A substance is said to “enhance” an immune response of a host organism to an antigen (i.e. is an adjuvant) if the immune response experienced by the host organism is greater when an antigen is applied to the host organism, in combination with the putative adjuvant, compared to the immune response experienced by the host organism when an antigen is applied without the putative adjuvant. Various immune cell assays can give a good indication of whether a substance is likely to be an effective adjuvant in a host organism or not (see for example, U.S. Pat. No. 6,406,705, which cites measuring the antibody forming capacity and number of lymphocyte subpopulations using a mixed leukocyte response assay and lymphocyte proliferation assay).

Different adjuvants can promote different types of response, for example, an inflammatory TH1 response or an antibody-dominated response. Some adjuvants, for example, pertussis toxin, stimulate mucosal immune responses, which are particularly important in defence against organisms entering through the digestive or respiratory tracts.

A TH1 immune response may be elicited using a TH1 adjuvant. In one embodiment, the adjuvant is a TH1 adjuvant. A TH1 adjuvant will elicit increased levels of IgG2a production relative to immunisation of the antigen without adjuvant. Alternatively, the adjuvant is a TH2 adjuvant.

The adjuvant of the third aspect of the immunogenic composition provided herein may include mineral containing compositions, such as aluminium salts, and ADP-ribosylating toxins and detoxified derivatives thereof, saponin formulations, virosomes and virus like particles, non-toxic derivatives of enterobacterial lipopolysaccharide (LPS) or CpG rich polynucleotides.

Kits

In the fourth aspect of the invention, there is provided a kit containing the immunogenic composition of any of the previous aspects of the compositions described herein. In one embodiment, the Francisella strain and an adjuvant are separate components of the kit. The advantage of having the Francisella strain and the adjuvant separated is that different buffer conditions or storage conditions can be imposed on the separate components in order to keep the both components in an optimum condition for administering to a host organism. Alternatively, the Francisella strain and adjuvant are present in a single composition. This has the advantage that having the components in a single composition results in reduced packaging and the cost savings inherent therein. In addition, if the components are in a single composition, this makes for ease of use compared to having the components separated and there is no danger of mixing the components in the wrong proportions.

In another embodiment, the pilin type IV antigen, or antigenic analog thereof, and the adjuvant are separate components of the kit. Alternatively, the pilin type IV antigen, antigenic analog thereof, are present in a single composition.

Preferably, the immunogenic composition is in a lyophilised form.

The kit may further include a second component including one or more of the following: instructions, syringe or other delivery device, further adjuvant, or pharmaceutically acceptable formulating solution.

The kit may alternatively contain a delivery device pre-filled with one or more of the immunogenic compositions described herein.

Medicaments and Routes of Delivery

In a fifth aspect of the immunogenic composition, an immunogenic composition of the first, second or third aspects of the invention, or a kit of the fourth aspect of the invention is provided for use as a medicament.

Preferably, the immunogenic composition, whether in the form of a single composition or separated into various components (e.g. Francisella strain and adjuvant), is in the form of a liquid (solution or suspension), a solid (including lyophilised compounds, a tablet, a capsule, or a dragee), a gas (including an aerosol e.g. an injectable aerosol or a spray), a gel or a cream.

The route of delivery into the host organism may include intradermal, transdermal, subcutaneous, intravenous, intramuscular, intraarterial, intramedullary, intrathecal, intraventricular, intraperitoneal, intranasal, enteral, topical, sublingual, vaginal, rectal, oral, aural, ocular or by scarifying.

For example, the immunogenic composition may be suitable for topical administration e.g. in the form of a spray, an aerosol, a gel, a cream, an ointment, a liquid, or a powder. The composition may be suitable for oral administration e.g. in the form of a dragee, a tablet, a capsule, a spray, an aerosol, a liquid e.g. a syrup, a tincture (particularly when the pharmaceutical composition is solubilized in alcohol). The composition may be suitable for aural or ocular administration e.g. in the form of drops or sprays. The composition may be suitable for pulmonary administration e.g. in the form of an aerosol, a spray or an inhaler. The composition may be suitable for rectal or vaginal administration e.g. in the form of a suppository (including a pessary). The composition may be suitable for subcutaneous, intramuscular or intradermal administration e.g. in the form of an injector and/or injection. Preferably, intradermal is by a high pressure jet injector. Gene guns or hyposprays may also be used to administer the immunogenic compositions of the invention.

The immunogenic composition described herein may be prepared in a solid form which is suitable for solubilizing or suspending in a liquid. Preferably, the liquid is water or alcohol. The solid form can be a lyophilised composition or a spray freeze-dried composition. The solid form can be solubilized or suspended in liquid immediately prior to administration. Advantages of using lyophilised compositions include economical savings because of cheaper transportation costs and easier storage conditions because the compositions tend to be more stable in a lyophilised state compared to being in solution. In such cases, the composition is preferably supplied as a kit (described above) that includes all or some of the components necessary for reconstitution into a form suitable for administration to the host. The kit may contain a mixture of forms, e.g. the antigen may be in a liquid form whereas the adjuvant may be in a lyophilised state. Alternatively, all the components of the kit may be in one type of form e.g. they are all in a lyophilised state.

When the immunogenic composition is lyophilised, preferably, a stabilising agent is added to the composition before lyophilization. Such a stabilising agent could be peptone. For reconstitution of the immunogenic composition for scarification, preferably, the immunogenic composition is reconstituted in a solution of 50% (volume per volume) glycerin in McIlvaine solution. If the lyophilised immunogenic composition is intended for injection, preferably saline is used for reconstitution.

The medicament of this aspect of the invention may be in the form of a prophylactic (e.g. as a vaccine) or a therapeutic for treating those host organisms that already have the disease.

The medicament may also include other components that help stabilise the immunogenic composition during storage or in vivo, post-administration to the host organism. Stabilising agents are well known in the art and include compounds such as peptone.

As used herein, a “pharmaceutically acceptable carrier” is a term understood in the art. Suitable carriers may be solid, liquid or gas, and they may suitably be formulated for administration via a particular route of delivery as described above. Compositions are suitably prepared in unit dosage forms, as conventional in the art. They are administered at dosages which are determined using clinical practice, and depend upon factors such as the nature of the patient, the severity of the condition, and the precise vaccine strain being employed.

Typically dosage units will comprise 10⁵-10⁸ cfu. Dosages may be boosted as appropriate or necessary.

Compositions may also contain further immunogenic reagents which are effective against F. tularensis or other diseases.

Vaccines

As explained above, there is a need for a new vaccine against Francisella tularensis. The present invention provides for the use of the immunogenic composition of the first, second or third aspects of the invention or the kit of the fourth aspect of the invention for the manufacture of a medicament, wherein the medicament is a prophylactic. Preferably, the prophylactic is a vaccine.

By “vaccine” is meant a substance containing antigenic material that can be used to stimulate the development of antibodies and thus confer immunity against one or more diseases.

Preferably, the vaccine is for prevention against tularemia. More preferably, the vaccine is suitable for vaccination of humans.

In another embodiment, the use of the immunogenic composition of the first, second or third aspect of the invention or the kit of the fourth aspect of the invention for the manufacture of a medicament wherein the medicament is for treating tularemia.

The immunogenic composition and methods of use described above will be further understood with reference to the following non-limiting examples.

### EXAMPLES

### Example 1

### Bacterial Strains, Plasmids, Growth Conditions and DNA Methods

The strains and plasmids used in these Examples are listed below in Table 2.

TABLE 2

| | | |
|---|---|---|
| | | Source/ |
| Strains/Plasmids | Genotype/phenotype | reference |

Strains
F. tularensis

| | | |
|---|---|---|
| FSC040* | Subsp. novicida ; ATCC 15482 | [Johansson, |
| | | 2000 #30] |
| FSC041* | Subsp. tularensis ; Vavenby | [Johansson, |
| | | 2000 #30] |
| FSC074* | Subsp. holarctica ; SVAT7 | |
| FSC155* | Subsp. holarctica ; Live Vaccine Strain; LVS; ATCC29684 | [Johansson, |
| | Russia | 2000 #30] |
| FSC200* | Subsp. holarctica ; 3001MA | [Johansson, |
| | | 2000 #19] |
| FSC237* | Subsp. tularensis ; SCHU S4 | [Broekhuijsen, |
| | | 2003 #4] |
| FSC338* | Subsp. holarctica ; Strain 015 | |
| FSC352* | Subsp. holarctica | |
| FSC354* | Subsp. holarctica | |
| FSC473* | FSC155/pAL10; FSC155 expressing the pilin gene (FTT0890) in | This study |
| | trans; Tc^{R} | |
| FSC498* | FSC354/pAL10; FSC354 expressing the pilin gene (FTT0890) in | This study |
| | trans; Tc^{R} | |
| FSC507* | FSC354/pil⁺; FSC354 expressing the pilin gene (FTT0890) in cis | This study |
| FSC476* | FSC155/pEMS10; FSC155 expressing the pilin gene (FTT0890) | This study |
| | and Flag epitope in trans; Tc^{R} | |
| FSC508* | FSC354/pEMS10; FSC354 expressing the pilin gene (FTT0890) | This study |
| | and Flag epitope in trans; Tc^{R} | |

E. coli

| | | |
|---|---|---|
| Top10 | F⁻ mcrA Δ(mrr-hsdRMS-mcrBC), Φ80lacZΔM15 ΔlacX74 recA1 | Invitrogen |
| | deoR araD139 (Δara-leu)7697 galU galK rpsL (Sm^{r}) endA1 | |
| | nupG | |
| S17-1λpir | recA, thi, pro, hsdR⁻M+, <RP4:2-Tc:Mu:Km:Tn7>Tp^{R}, Sm^{R} | [Simon, 1983 |
| | | #14] |

P. aeruginosa

| | | |
|---|---|---|
| PAK | Wild type | D. Bradley |
| PAKpiLA⁻ | PAK, pilA in frame deletion | [Kagami, |
| | | 1998 #12] |
| PAKpSpil⁻ | PAKpiLA⁻ expressing the synthetic pilin gene (FTT0890) in trans; | This study |
| | Gm^{R} | |

Plasmids

| | | |
|---|---|---|
| pCR ® 2-TOPO | TOPO-cloning vector. Amp^{R}, Km^{R} | Invitrogen |
| pCR ® 4.0-TOPO | TOPO-cloning vector. Amp^{R}, Km^{R} | Invitrogen |
| pUC18 | Cloning vector. Amp^{R} | (Norrander) |
| pDM4 | Suicide plasmid. sacB; mobRP4; onR6K; Cm^{R} | [Milton, 1996 |
| | | #11] |
| p34S-Gm | Cloning vector carrying a Gm resistance cassette, Gm^{R} | [Dennis, 1998 |
| | | #10] |

The primers used in these Examples are shown in Table 3.

TABLE 3

| | | Relevant |
|---|---|---|
| | | restriction |
| | | enzyme |
| Primer | Primer sequence^{a} 5′-3′ | site |
| pilAcomp_1F | GCATGTCATATGAAAAAGAAAATGCAAAAAGGT | NdeI |
| pilAcomp_1R | GCATGTGAATTCGATTAGATAGCATTACAGTTAGA | EcoRI |
| pilAcomp_4F | CAACGCCAAAGATTACATCAC | |
| pilAcomp_4R | TTCTGTCATTCCTGACTCGAC | |
| pilA1flag_1R | GCATGTGAATTCTTATTTATCATCATCATCTTTAT | EcoRI |
| | AATCGATAGCATTACAGTTAGATGGT | |
| pilA1-5′ | CCAAATCTAGACATGTACTCTAACTACACTACA | XboI |
| pilA1-3′ | AGATTACTCGAGTTATTAGATAGCATTACAGTTAGA | XhoI |
| A | TTCTCACTAGTTGAGTTAATG | |
| B | CCATTAGCTCAACTAGTGAGAA | |
| C | GGGGTAGTACTTTAAATCCT | |
| D | GCATCACACGAGAGGCATT | |
| E | GTGCCTTTGATCGTGCTTAC | |
| F | CTTGAGATGCTGATACACCT | |

^{a}Restriction sites are underlined.

F. tularensis strains were grown on modified Thayer-Martin agar plates (Sandström, G., Tärnvik, A., Wolf-Watz, H. & Löfgren, S. (1984) Infect. Immun. 45, 101-106) at 37° C. in 5% CO₂ or in Chamberlain medium (Chamberlain, R. E. (1965) Appl. Microbiol. 13, 232-235). E. coli and P. aeruginosa strains were grown on blood agar base (BAB; Merck) plates, on minimal medium plates (Vogel, H. J. & Bonner, D. M. (1956) J. Biol. Chem. 218, 97-106) (only P. aeruginosa), or in Luria-Bertani broth (LB). Where appropriate, antibiotics were used at the following concentrations; kanamycin 50 μg/ml, tetracycline 10 μg/ml, chloramphenicol 5 μg/ml (F. tularensis strain FSC354), or 25 μg/ml (E. coli), carbenicillin 60 μg/ml (P. aeruginosa), and gentamicin 20 μg/ml (P. aeruginosa). Preparation of plasmid DNA, restriction enzyme digests, ligations and transformations into E. coli were performed essentially as described by Sambrook et al., (Sambrook, J., Fritsch, E. G. & Maniatis, T. (1989) in MOLECULAR CLONING: A LABORATORY MANUAL, 2 ED. (Cold Spring Harbor Laboratory Press, New York)). Generally the primers (Table 3) were constructed from the SCHU S4 and LVS genome. Before sub-cloning DNA fragments into a suitable vector, the PCR fragments were first cloned into vector pCR®2.1 or pCR®4.0-TOPO cloning vector (Invitrogen AB, Stockholm, Sweden), and verified by sequencing (MWG Biotech, Ebersberg, Germany). Positive clones were verified by PCR and when possible, mutant strains were confirmed also by Western blot analysis using a polyclonal rabbit antiserum raised against the pilin gene product (FTT0890).

### Example 2

### Cis and Trans Complementation of the Pilin Gene (FTT0890) in Strain FSC354

For complementation in trans, vector pKK214GFP was used. The complete pilin gene from FSC352 was amplified with primers PilAcomp-1F and PilAcomp-1R. The PCR-product was sub-cloned as an Nde1/EcoR1 410 bp fragment into pKK214GFP in frame with the stress induced groESL promoter (constitutively on at 37° C.) and denoted pAL10. pAL10 was then introduced into strain FSC354 by cryotransformation (Pavlov, V. M., Mokrievich, A. N. & Volkovoy, K. (1996) FEMS Immunol. Med. Microbiol. 13, 253-256) and following selection with tetracycline. The cis-complementation construct was generated by PCR using FSC352 as template and primers pilAcomp-4F and pilAcomp-4R. This resulted in a 2441 bp fragment covering the pilin gene (FTT0890) and approximately 1 kb flanking sequence on each side of the gene. The fragment was inserted into the suicide mutagenesis vector, pDM4 (Xho1-Sac1), resulting in vector pAL11, and transformed into E. coli S17-1λpir. Complementation of strain FSC354 in cis was done by conjugal mating experiments with E. coli S17-1λpir as the donor strain followed by sucrose selection, essentially as previous described by Goloviliov et al., (Golovliov, I., Sjostedt, A., Mokrievich, A., & Pavlov, V. (2003) FEMS Microbiol. Lett. 222, 273-280).

### Example 3

### Construction of the Pilin Gene (FTT0890) Containing a C-Terminal Flag-Epitope

The pilin gene and a C-terminal Flag-epitope, DYKDDDDK, were cloned under the groESL promoter of pKK214GFP by using primers pilAcomp-1F and pilA1flag-1R in a PCR with strain FSC352 as template. The amplified Nde1/EcoR1 product was subcloned into pKK214GFP, generating vector pEMS10, and transformed into E. coli TOP10 One shot chemically competent cells (Invitrogen). pEMS10 was then introduced into the recipient FSC354 strain by cryotransformation (Pavlov, V. M., Mokrievich, A. N. & Volkovoy, K. (1996) FEMS Immunol. Med. Microbiol. 13, 253-256).

### Example 4

### Production of Polyclonal Antiserum

Based on the DNA sequence derived from F. tularensis SCHU S4 genome, the hydrophilic C-terminal region of the putative pilin gene (FTT0890) was amplified using primers PilA1-5′ and PilA1-3′. The resulting 317-bp sequence was cut by using the unique flanking Xba1 and Xho1 sites and cloned in frame into the expression vector pGEX-KG with an N-terminal glutathione S-transferase (GST) tag. The recombinant plasmid was transformed into E. coli DH5α. The GST fusion protein, was purified by glutathione affinity chromatograph (GSTrap and Glutathione Sepharose 4 Fast Flow, Amersham Pharmacia Biotech) for subsequent use as an immunogen for polyclonal rabbit antiserum production by AgriSera AB, Vännäs, Sweden.

### Example 5

### Design of a Synthetic F. tularensis Pilin Gene (FTT0890) for Expression in P. aeruginosa (PAK)

The amino acid sequence of the F. tularensis pilin gene (FTT0890) was converted into a DNA sequence with optimized codon usage for maximal protein expression in P. aeruginosa. A 645 bp sequence, including a 408 bp sequence encoding the F. tularensis pilin gene, the ribosome binding site and the terminator for the pilA gene from P. aeruginosa pilA, was chemically synthesized (GenScript Corp., New Jersey, USA). The synthesized gene was delivered into the EcoR1-BamH1 site of pUC18 resulting in pKK219. To facilitate the subsequent selection in P. aeruginosa, a gentamicin (Gm) cassette derived from Sal1-digested p34S-Gm was cloned into the unique Sal1 site of pKK219 generating pKK220. The EcoR1-HindIII fragment was ligated into pMMB66EH and transformed into S17-1λpir which was used as the donor strain in conjugal experiments with P. aeruginosa. After introduction of the plasmid to P. aeruginosa PAKpilA the strain was analyzed for expression and secretion of the pilin gene product.

### Example 6

### Expression and Secretion of F. tularensis Pilin Protein by P. aeruginosa pilA-Mutant

Overnight cultures grown at 37° C. in LB were diluted 100 fold in the same medium, grown for 1 hour prior to addition of IPTG to a final concentration of 0.5 Mm and grown for an additional 3 hours. Whole cell lysates were analyzed by immunoblotting using a polyclonal rabbit antiserum.

### Example 7

### Detection of Cell Surface Pili

Francisella were grown overnight on plates and resuspended in phosphate-buffered saline (PBS) and vigorously shaken by vortexing. The suspension was centrifuged twice at 13 000×g, five minutes each time. The pilus-enriched supernatant was incubated at 65° C. to kill any remaining bacteria and the pili were allowed to aggregate at 4° C. for 18 hours and then ultracentrifuged at 150 000×g for one hour. The resulting pellet was resuspended in sample buffer (12.5% glycerol, 0.1 M Tris HCl [pH 6.8]) lacking SDS and mercaptoethanol and loaded on a 12% SDS gel without boiling. The proteins loaded on the gel were derived from the same starting amount of bacteria.

### Example 8

### RNA Isolation and Reverse Transcription (RT) PCR

Bacteria were grown for 18 hours on agar plates and resuspended in PBS to an optical density at 540 nm of 1.0. Total RNA was extracted from 0.5 ml of suspension of each strain by the use of TRIzol reagent (TRIzol™ Reagent, Life Technologies) as instructed by the manufacturer. RNA was treated with RNase-free Dnase I (Roche), phenol extracted, and ethanol precipitated. About 3 μg RNA from each preparation was used to synthesize cDNA by the use of random hexamers (final concentration 25 ng/μl) and Superscript II enzyme (Life Technologies). The integrity of the DNAse 1-treated total RNA was verified by setting up negative control tubes containing experimental cDNA without RT enzyme.

### Example 9

### Gel Electrophoresis and Western Blotting

Samples were boiled in the presence of SDS and mercaptoethanol for 5 minutes, or in the absence of SDS and mercaptoethanol and not boiled, and then applied on a uniform 12% acrylamide gel for electrophoresis as described by Laemmli (Laemmli, U. K. (1970) Nature 227, 680-685). Proteins were transferred to Immobilon-P Transfer Membranes (Millipore) membrane using Trans-Blot Semi-Dry transfer cell (Bio-Rad). Membranes were blocked overnight in Tris-buffered saline (TBS) with 5% nonfat dry milk. Membranes were probed with a polyclonal rabbit antiserum. When a horseradish peroxidase-conjugated secondary antibody system was used, filters were developed by using the ECL Kit (Amersham Pharmacia Biotech). When using an alkaline phosphatase-conjugated (Roche) secondary antibody system the visualization was accomplished by treating the blots with 0.1% (w/v) NBT (Nitro-blue-tetrazolium; Sigma Chemical Company, St. Louis, Mo.) and 0.05% (w/v) BCIP (5-bromo-4-chloro-3-indolyl phosphate; Sigma).

### Example 10

### Mice Experiment

For the infection study, 6 to 9 week old C57BL6 female mice were used (Scanbur BK AB, Sollentuna, Sweden). Mice were housed under conventional conditions, given food and water ad libitum, and were allowed to acclimatize for at least seven days before use. The study was approved by the Local Ethical Committee on Laboratory Animals in Umeå, Sweden. F. tularensis strains were grown 16 hours on a plate before suspending the bacteria in PBS (pH 7.4) to an OD₅₄₀ of 1, ˜2×10⁹ bacteria/ml. The culture was then diluted in PBS into doses ranging from 1 to 10⁷ bacteria in a total volume of 100 μl, followed by subcutaneous injection at the groin-area of the mice. To confirm the infection doses, the diluted cultures were controlled by viable count. The infected mice were observed two to three times a day for 14 days to follow the infection course and they were sacrificed if they showed severe sickness. Typical symptoms of tularemia in mice are a hunched carriage and tousled fur. To verify the stability in vivo of the expression vector pAL10, bacteria were recovered from the spleen eight days after infection and analyzed by viable count, plasmid preparation followed by digestion of the cloned insert, PCR, and Western blot.

One region encoding three genes with the typical signatures for a type IV pilin (FIG. 1A) was particularly interesting since the first gene mapped in a region that is flanked by 120 bp repeats. Comprehensive studies of the Francisella genome sequence revealed more than 70 regions that are flanked by long direct repeats of DNA. Comparisons of strains isolated from different tularemia outbreaks revealed that some strains lacked the pilin gene. However, analysis of a large number of strains showed that in most type B strains isolated from outbreaks of tularemia in Sweden, like strain FSC200, the first gene which was flanked by the direct repeats was intact while the two downstream pilin-like genes appeared to be non-functional due to non-sense mutations (FIG. 1A). Interestingly, one isolate, FSC074, contained a mixture with respect to presence of the first pilin gene. PCR analysis of single colonies revealed that in some colonies the pilin gene was intact while the gene apparently had been deleted in others. One colony of each type was purified and the variant with the intact gene was denoted FSC352 and the one where the pilin gene had been deleted was denoted FSC254. When the region encoding the pilins were subjected to DNA sequencing, one could verify that the deletion of the pilin gene in strain FSC354 had occurred by a mechanism involving homologous recombination mediated by the direct repeats, leaving one chimeric repeat in strain FSC354 (FIG. 1C). The deletion of the pilin gene in strain FSC354 results in an in-frame fusion between the 5′ part of the first pilin gene FTT0890 and the downstream pilin-like FTT0889. The hybrid gene resulting from the fusion retains an intact N-terminal signature common for the type IV pilins. However, as the downstream gene contains two different non-sense mutations, the resulting hybrid gene is not likely to be functional (FIG. 1A).

### Example 11

### Genetic Analysis of a Type IV Pilin Gene Cluster

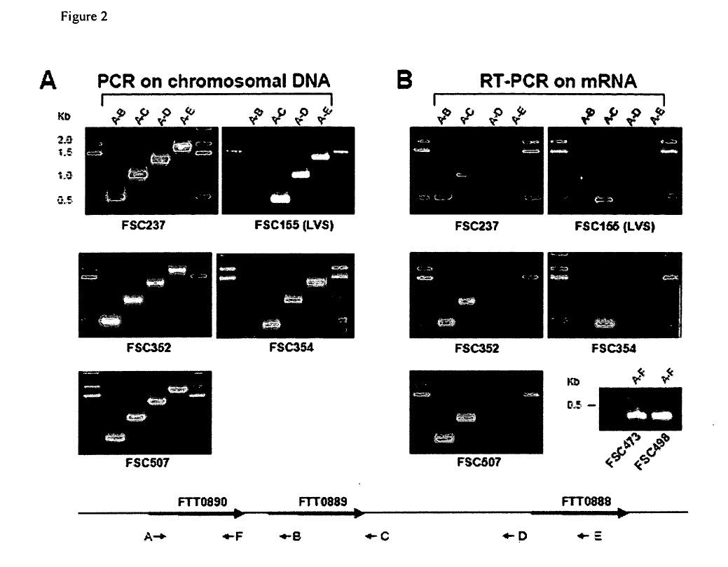

In this Example, transcription and expression of pilin gene FTT0890 was performed. In order to establish if the pilin genes were transcribed, RNA was prepared from the different strains. It could be verified that all the pilin genes encoded by the operon were transcribed in all strains including strain FSC354 where the transcript, however, was shorter as a result of a deletion event (FIG. 2B). RT-PCR on mRNA revealed that the first two pilin genes, FTT0890 and FTT0889, are transcribed from a common promoter and expressed by SCHU S4 and FSC352 (FIG. 2B). The third pilin gene, FTT0888 is most likely expressed from a promoter mapping in the intragenic region between FTT0889 and FTT0888 as no mRNA encoding all three pilin genes could be detected (FIG. 2B). By using primers mapping just upstream and downstream of FTT0888, the inventors could also verify that his gene was transcribed in the investigated strains (data not shown).

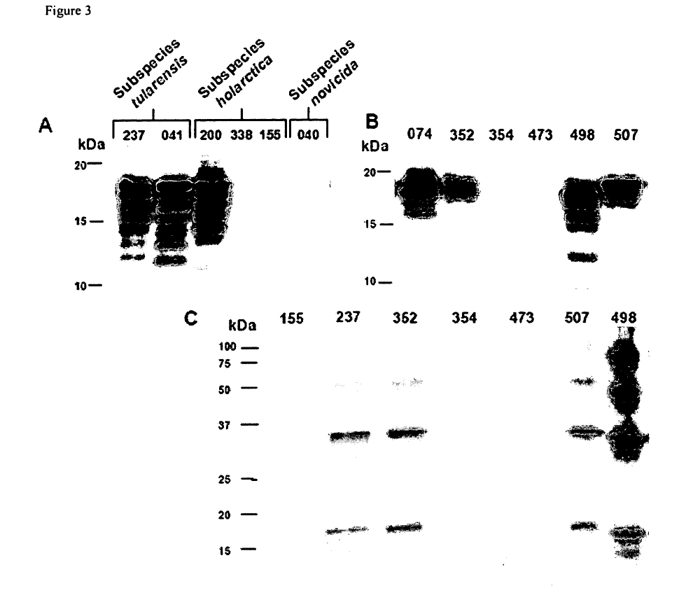

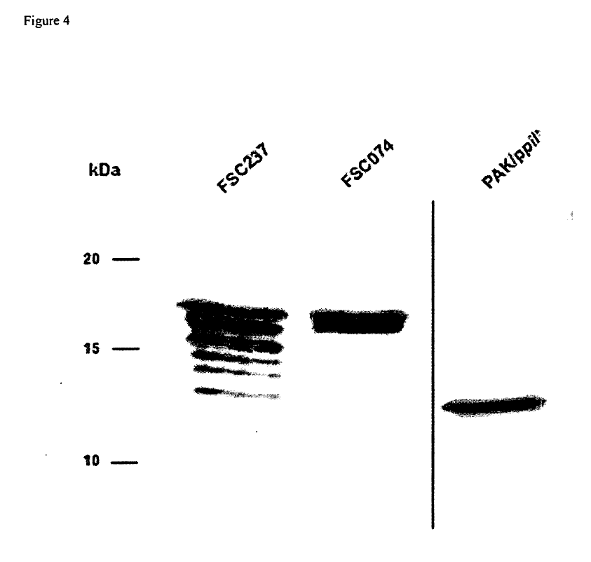

To facilitate studies of pilin protein expression, the pilin encoded by gene FTT0890 was expressed and purified as a GST-fusion protein which was subsequently used to generate polyclonal antibodies. Western blot analysis verified that the pilin was expressed in all strains analyzed except in the three subspecies Holartica strains FSC354, FSC338, FSC155 (LVS), and the subspecies novicida strain FSCO40 (FIGS. 3A and 3B). The apparent size of the pilin protein was about 18 kD which is about 4.5 kD larger than expected from the size of the protein encoded by gene FTT0890 and the protein also appeared heterogeneous in size indicating either degradation or post-translational modification (FIGS. 3A and 3B). Several pathogens including Neisseria species and several strains of P. aeruginosa are known to modify the Type IV pilin by glycosylation (Kus, J. V., Tullis, E., Cvitkovitch, D. G. & Burrows, L. L. (2004) Microbiology 150, 1315-1326). In order to establish if the putative type IV pilin of Francisella was modified, the pilin gene was expressed in the heterologous host P. aeruginosa PAK. Strain PAK is known not to glycosylate its type IV pilin (Kus, J. V., Tullis, E., Cvitkovitch, D. G. & Burrows, L. L. (2004) Microbiology 150, 1315-1326). The heterologous pilin gene was expressed (FIG. 4) and secreted (data not shown) by P. aeruginosa. Importantly, the pilin now appeared as an approximately 13.5 kD protein which is in accordance with the expected size and in this case the protein also appeared as a homogeneous band (FIG. 4). These results indicate that the putative Type IV pilin of Francisella is post-transcriptionally modified possibly by glycosylation as described for Type IV pilin of other pathogens.

### Example 12

### The Pilin Protein (FTT0890) is Exported to the Cell Surface where it Multimerises

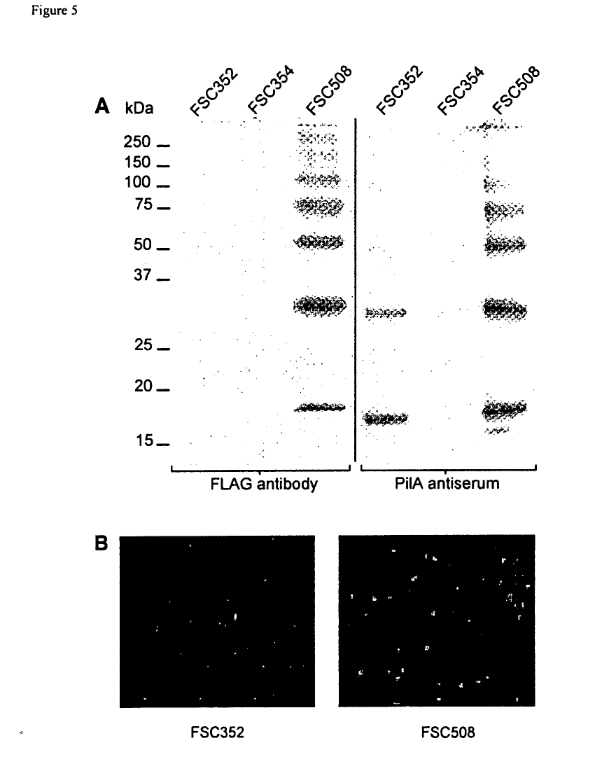

The genome sequence of SCHU S4 encodes a number of putative type IV pilins and an experiment was needed to establish if the pilin protein (FTT0890) was exported by F. tularensis and potentially also assembled into a polymeric structure. Sheared fractions of different bacterial strains were analyzed by Western blot and thereby the inventors could verify that the pilin protein was exported. When the Western blot analysis was conducted under non-denaturing conditions the pilin protein appeared as a ladder of bands with a size difference between the individual bands corresponding to the size of the pilin (FIG. 3C). This verifies that the pilin protein is secreted and/or assembled at the bacterial surface where it forms multimers that are most likely are part of a Type IV pilin. In addition, to visualize export of the pilin to the cell surface, the pilin gene (FTT0890) from FSC352 was tagged at the C-terminal end with a Flag-tag. The tagged pilin was expressed in trans in strain FSC354 (lacking the pilin) and expression was monitored by Western blot, using antibodies raised against the Flag-tag and the pilin. This analysis confirmed that the Flag-tagged pilin was expressed and could similar to the untagged pilin in FSC352 also multimerises (FIG. 5A). The strain expressing the Flag-tagged pilin (FSC508) was also analyzed by fluorescence microscopy using the anti-Flag antibodies. Strong immunofluorescence of the bacterial surface was seen for the strain expressing the Flag-tagged pilin (FIG. 5B). This further strengthens our finding that the type IV pilin is exported to the bacterial cell surface. The immunofluorescence microscopy does not reveal any filament like structures extending from the bacterial surface which indicates that either this pilin protein is not the major structural component of the putative type IV pilin of F. tularensis or that the Flag-tag interferes with filament formation.

### Example 13

### The Pilin is not Required for Intracellular Replication

Next, an experiment was devised to address the significance of the pilin with respect to virulence of F. tularensis. Recent studies have confirmed that intracellular survival and replication is an important virulence mechanism common to several subspecies of F. tularensis (Golovliov, I., Sjostedt, A., Mokrievich, A., & Pavlov, V. (2003) FEMS Microbiol. Lett. 222, 273-280; Lauriano, C. M., Barker, J. R., Yoon, S. S., Nano, F. E., Arulanandam, B. P., Hassett, D. J., & Klose, K. E., (2004) Proc. Natl. Acad. Sci. USA 101, 4246, 4249; Nano, F. E., Zhang, N., Cowley, S. C., Klose, K. E., Cheung, K. K., Roberts, M. J., Ludu, J. S., Letendre, G. W., Meierovics, A. I., Stephens, G., & Elkins, K. L. (2004) J. Bacteriol. 186, 6430-6436) When the ability of strains FSC352 and FSC354 to multiply in macrophage-like cell lines of human and mouse origin was investigated, it was found that the presence of pilin did not influence the ability to infect, survive and multiply inside the macrophage-like cell lines of both mouse (J774.1) and human (THP) origin (data not shown). This is perhaps not too surprising as intracellular replication has been established for both subspecies novicida and subspecies Holarctica, vaccine strain, LVS, which both do not express the pilin gene. This indicates that the type IV pilin has no direct role in the ability of Francisella to infect and multiply in phagocytes. It was also investigated if expression of the pilin influenced binding to other human cells but found that Francisella strains adhered to epithelial cells, like HeLa cells, equally well irrespective of pilin gene expression (data not shown).

### Example 14

### The Pilin is Essential for Virulence of F. tularensis

Finally, an experiment was devised to address whether the pilin protein played a critical role in an animal infection model. Infection of mice is the most frequently used model for tularemia infections. One problem of using this model to predict virulence in human infections is that the type B vaccine strain, LVS, is highly virulent via several infection routes in mice (Conlan, J. W., KuoLee, R., Shen, H. & Webb, A. (2002) Microb. Pathog. 32, 127-134). However, if mice are infected via a subcutaneous route, the vaccine strain is severely attenuated compared to virulent type A and type B strains. First, it was decided to study if there was any selection for either of the genotypes of the original isolate FSC074, which was a mixture of FSC352 and FSC354. Mice were infected subcutaneously with a mixture of FSC352:FSC354 in a ratio of 10:90 using different infection doses. Bacteria were recovered from spleens and livers of the infected animals and almost exclusively (95%) only strain FSC352 could be recovered (data not shown). This indicates that there is a strain selection for the pilin positive variant in animals and indicates that pilin negative strain is highly attenuated.

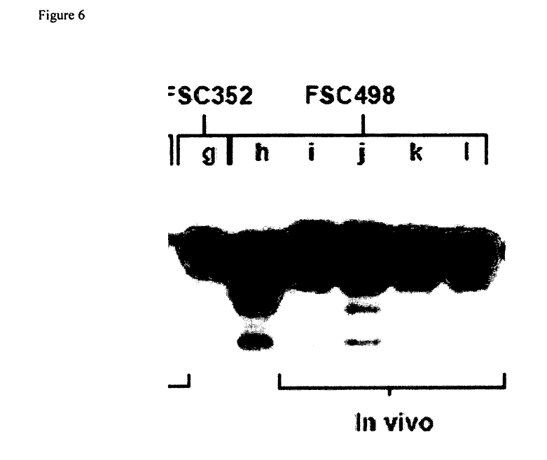

To conclusively resolve the issue of the importance of the pilin gene for virulence the positive and negative strains, FSC352 and FSC354, were compared in their ability to infect mice via the subcutaneous route. It was found that the pilin negative strain FSC354 was severely attenuated (Table 4: FIG. 6).

TABLE 4
Dose of F. tularensis strains required to induce lethal infection
in C57 BL mice

| Strain | Cfu |
|---|---|
| FSC352 | <10* |
| FSC354 | 2, 3**, 10⁷** |
| FSC507 | <10* |
| FSC498 | 1, 2**, 10⁶** |

*The Cfu value is a LD₅₀ calculation (Reed, L. J. & Muench, H. (1938), Am. J. Hyg. 27, 493-497) but should not be seen as a standard LD₅₀ experiment since mice were sacrificed if they showed severe sickness.
**Less than 10 bacteria are required to induce lethal infection. The low infection dose makes a LD₅₀ calculation less significant and is therefore not done.

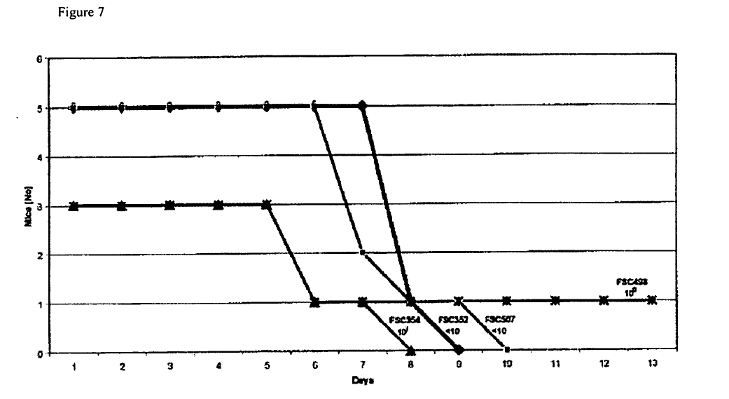

The infection doses required to cause lethal infection differed by a factor greater than 105. Even if the two variants FSC352 and FSC354 were found in the same isolate, the two strains could not be formally regarded as isogenic pairs different only at the pilin locus. It was therefore decided to complement the pilin negative strain FSC354 with a plasmid expressing the pilin gene from FSC352 in trans. Few promoters have so far been identified and characterized in F. tularensis but in a recent study a plasmid containing the promoter region of the groESL gene was successfully used to complement an iglC mutant (Lai, X. H., Golyliov, I. & Sjöstedt, A. (2004) Microb, Pathog. 37, 225-230). The advantage of the groESL promoter is that it is known to be expressed under in vivo like conditions (Lai et al., supra). The pilin negative strain FSC354 was therefore complemented in trans by expressing the pilin gene under the control of the groESL promoter in the shuttle vector pKK214GFP (Table 2). First it was needed to establish that the pilin was expressed in vitro and it was found that introduction of plasmid restored pilin expression to levels similar to those of the pilin positive strain FSC352 (FIG. 3B). Analysis of sheared fractions indicated that the pilin was overexpressed in the trans-complemented strain FSC498 compared to strain FSC352 (FIG. 3C). When the trans-complemented strain was used to infect mice, complementation was incomplete and the dose required to establish lethal infection was only about 10 times lower compared to the pilin negative strain FSC354 (Table 4, FIG. 6). Colonies isolated from spleens of infected mice revealed that the plasmid was stably maintained during infection and the colonies isolated from the mice showed similar pilin expression levels as observed in vitro prior to infection (FIG. 7). This indicates that the lack of complementation was either on the level of in vivo regulation of pilin expression or that the higher levels of pilin expression impaired function of the Type IV pilin in vivo. The inventors therefore decided to complement the pilin gene deletion by a construct where the pilin gene was expressed from its native promoter. By complementing the pilin in cis, i.e. by recombining the pilin gene into the chromosome by a double cross-over event, the inventors achieved a complementation where the pilin was under control of its native promoter as well as in single copy gene dose. The cis-complemented strain showed similar virulence properties as strain FSC352 (table 4, FIG. 6), verifying that the attenuation of strain FSC354 was really a direct result of detection of the pilin gene and that a functional pilin is likely to be critical for Type IV pilin function as well as for virulence in mice by peripheral routes of infection.

### Example 15

### Analysis of Results

These experiments describe the implication of an adhesin in the virulence of F. tularensis for the first time. Interestingly, the level of attenuation is similar as seen for the vaccine strain LVS which is also highly attenuated by subcutaneous infections in mice. Here, it is important to note that also in LVS the pilin gene (FTT0890) has been deleted by an event mediated by the flanking direct repeats (unpublished results). The deletion event is likely to occur with fairly high frequency during in vitro culture when there is no selection to maintain the gene. The deletion event is also likely to occur in response to stress or starvation, procedures which previously have been frequently used to attenuate bacterial strains to make them suitable as live vaccines. Even if the level of attenuation for loss of the single pilin gene of strains FSC354 and LVS was similar, it is still possible that LVS has additional mutations. This is supported by preliminary results which show that it is not possible to cis-complement LVS by introduction of the pilin gene. However, in a trans-complemented LVS strain, the pilin gene is transcribed while protein expression levels are very low. This suggests that additional genes, important for export/assembly of type IV pilin are either not functional or not expressed. However, it is important to note that in the specific case of the pilin gene, the loss of the gene is irreversible and there is no risk for reversion of this particular attenuation now identified to have occurred in LVS. The genome of the type A strain SCHU S4 contains at least six putative type IV pilin genes, which appear fully functional. In type B strains all these putative genes may not be functional as some of the have mutations which result in premature stops of translation, as seen in this study for genes FTT0889 and FTT0888. This opens the interesting possibility that type A and type B strains express type IV pilin with different properties and that these properties may also reflect the differences in virulence seen between the two biovars of F. tularensis. In the type B strains the pilin encoded by FTT0890 appears to be required for a functional pili and it remains to see whether this is also the case for type A strains. It is important to note that the subspecies novicida has retained the pilin gene flanked by the direct repeats (Svensson, K., Larsson, P., Johansson, D., Byström, M., Forsman, M. & Johansson, A. (2005) J. Bacteiol., accepted for publication in the June issue), but in this case the pilin gene is not homologous to FTT0890 as reflected by a negative Western blot using antiserum raised against the pilin from the type A/B strains. The difference has also been verified by DNA sequencing (data not shown) and the presence of a different pilin gene could explain the lower virulence of subspecies novicida even though in the mouse infection model subspecies novicida is somewhat more virulent compared to LVS (Kieffer, T. L., Cowley, S., Nano, F. E. & Elkins, K. L. (2003) Microbes Infect. 5, 297-403). Altogether the findings provided herein support that the pilin gene expressed by virulent type A and type B strains is crucial for expression of a type IV pilin with an important role in virulence.

So far, the inability to show that the pilin studied herein contains the structural component of the type IV pilin has been unattainable. Attempts to use the antibody generated against a GST-fusion of the pilin in surface labeling of the bacteria have been unsuccessful and it is possible that this antibody does not recognize the pilin when part of the filament structure. The Flag-tagged variant of the pilin was verified to be expressed, exported and capable of forming multimers but was still not possible to identify as part of a filament structure. It is possible that introduction of the Flag-tag somehow interfered with filament formation or that the pilin is essential for pilin formation/function without actually being the major pilin subunit as has been found for Type IV pilins of for instance Neisseira. Overall attempt to verify that the pilin is crucial for formation of type IV pilin in Francisella have been hampered by the very few pili seen expressed in vitro both on solid media and in liquid culture (data not shown). By growing very large numbers of bacteria, material has been sheared off that can be visualized as pili by electron microscopy. Here is seen a strict correlation with presence of the pilin gene and presence of pili further supporting that the pilin is crucial for functional type IV pilin in Francisella.

All references cited herein are hereby incorporated by reference.

Modifications and variations of the present immunogenic composition and methods of use will be obvious to those skilled in the art from the foregoing detailed description. Such modifications and variations are intended to come within the scope of the appended claims.

1. An immunogenic composition comprising a modified strain of Francisella species in combination with a pharmaceutically acceptable carrier, wherein the strain has inactivated cell surface pili.

2. The immunogenic composition of claim 1, wherein the strain has no pili exhibited on the cell surface.

3. The immunogenic composition of claim 1 wherein the Francisella strain is from a type A or type B Francisella species.

4. The immunogenic composition of claim 1 wherein the strain is a strain of Francisella tularensis.

5. The immunogenic composition of claim 1 wherein the strain is a strain of Francisella tularensis subspecies tularensis or a strain of Francisella tularensis subspecies novicida.

6. The immunogenic composition of claim 1 comprising a strain of Francisella having a deletion or mutation in a type IV pilin gene.

7. The immunogenic composition of claim 6 wherein the type IV pilin gene deletion or mutation occurs in a gene sequence selected from the group consisting of gene ID 3190704, ID 3191339, ID 3191481, ID 3191727, ID 3191703, ID 3191704, ID 3191700, ID 3191692, ID 3192032, ID 3191922, ID 3192189, ID 3191514, ID 3191399, ID 3191034, and ID 3191614.

8. The immunogenic composition of claim 6 wherein the deletion or mutation in the type IV pilin gene occurs in one or more of the type IV pilin FTT0888 gene, FTT0889 gene or FTT0890 gene sequences.

9. The immunogenic composition of claim 6, wherein the type IV pilin gene deletion or mutation results in an antigenic analog of a naturally occurring Francisella polypeptide.

10. The immunogenic composition of claim 9, wherein the naturally occurring Francisella polypeptide is a polypeptide from the SCHU S4 strain of F. tularensis.

11. The immunogenic composition of claim 10 wherein the naturally occurring Francisella polypeptide comprises a sequence selected from the group consisting of GI56707266, GI56707387, GI56707803, GI56707967, GI56707989, GI56707990, GI56707991, GI56708006, GI56708142, GI56708209, GI56708210, GI56708227, GI56708228, GI56708229 and GI56708230.

12. The immunogenic composition of claim 1, wherein the Francisella strain present in the immunogenic composition is a live strain wherein a gene has been inactivated, and the strain is able to produce a protective immune response when administered to an animal.

13. An immunogenic composition comprising a type IV pilin antigen, or an antigenic analog thereof, in combination with a pharmaceutically acceptable carrier.

14. The immunogenic composition of claim 13, wherein the pilin antigen is a Francisella pilin antigen or an antigenic analog thereof.

15. The immunogenic composition of claim 14 wherein the Francisella pilin antigen is from a Francisella type A or type B species or is an antigenic analog thereof.

16. The immunogenic composition of claim 14, wherein the Francisella pilin antigen is from a Francisella tularensis strain.

17. The immunogenic composition of claim 16, wherein the pilin antigen is from the SCHU S4 strain of F. tularensis.

18. The immunogenic composition of claim 17, wherein the pilin antigen is a polypeptide comprising a sequence selected from the group consisting of GI56707266, GI56707387, GI56707803, GI56707967, GI56707989, GI56707990, GI56707991, GI56708006, GI56708142, GI56708209, GI56708210, GI56708227, GI56708228, GI56708229 and GI56708230, and antigenic analogs thereof.

19. The immunogenic composition of claim 18, wherein the pilin antigen is a polypeptide comprising a sequence selected from the group of the type IV pilin FTT0888, FTT0889 or FTT0890 polypeptide sequences.

20. An immunogenic composition comprising a polynucleotide encoding a type IV pilin antigen or an antigenic analog thereof.

21. A method of preventing or treating infection by a Francisella species, comprising administering to a host organism an effective amount of the immunogenic composition comprising a modified strain of Francisella species in combination with a pharmaceutically acceptable carrier, wherein the strain has inactivated cell surface pili, or comprising a type IV pilin antigen, or an antigenic analog thereof, in combination with a pharmaceutically acceptable carrier.


SEQUENCE LISTING (26 sequences)

SEQ ID NO: 1 - DNA, 597 bp, Francisella tularensis
atgataactgtaaaatcaaaaaaaggtttttcacttactgagttacttgttgttatagctattataggcattttgtcgatactagttatcccagcgtattcaaactatgtcactagaacaagattgacagaagccttgacagttttagatggttataaaaaagatgtccaatcttattttattgcgcatggtgtctcaacccaagaacagttagaaaattatgatgtcacagatcatgctgatgttggtagcgatactactagcgtaatgagtaatatagtaggtcataacggtagaatagttggtgtaagcacgatcaaaggcacaacttaccaaatagctctaactccaaggatagagggtggtacattcaactggacatgctctatttcaatagttgaacaagacgctagctttgataatagttataatggtcaagtcttctttggatttattgataataatagtggctcatatgccgctccaagcagtagtatgctaccaagaggttgtaatgctacagatagcaaccaaaatactgatttcgaagcatatagatcagaaagacagacactagataacagagcttatgaatag

SEQ ID NO: 2 - DNA, 441 bp, Francisella tularensis
atgcaaacacacaaacaaacaggtttctcactagttgagctaatggtagtaattgcaataattgcaatcttagcagctgtagcaataccgatttactcaagctacaaagagcgtgctgcaattatcgaatctatgaacataattggtaatgtcaaagctagtatccaaaatgatatcaataataatctagatatctcccagcaaacttatgacacccctactggagtcactgtaacaggtagtacgtcaggagctactattgatataaatctaagccaaacttcaccacaacactttaccaacgataatgatattatcagacttagtggtgtagttgttagtggtagcactttccaatggacttgctcacataatgtcaacgcctcaacattaacagctagtaatgtcccacacacttgctcaagtacttttagtgcttag

SEQ ID NO: 3 - DNA, 408 bp, Francisella tularensis
atgaaaaagaaaatgcaaaaaggtttctcactagttgagttaatggtagtgatcgcgatcatcgctatcctagcagctgtagcgatcccgatgtactctaactacactacacgtgctcagttaggctctgatctatctgctctaggtggtgctaaagctacagtagctgaaagaatagctaacaacaatggtgatgcatctcaagttacaattcttcaagctaatgccgctgcaaatggtcttccaagtggtgcttcagttgctgctggtactattagttatccatcaacagtatctggtgcaacaattcaattagctcctacagtaagttccggtgctattacttggacttgtaatatttcaggtgtatcagcatctcaagtaccatctaactgtaatgctatctaa

SEQ ID NO: 4 - protein, 198 aa, Francisella tularensis
MITVKSKKGFSLTELLVVIAIIGILSILVIPAYSNYVTRTRLTEALTVLDGYKKDVQSYFIAHGVSTQEQLENYDVTDHADVGSDTTSVMSNIVGHNGRIVGVSTIKGTTYQIALTPRIEGGTFNWTCSISIVEQDASFDNSYNGQVFFGFIDNNSGSYAAPSSSMLPRGCNATDSNQNTDFEAYRSERQTLDNRAYE

SEQ ID NO: 5 - protein, 146 aa, Francisella tularensis
MQTHKQTGFSLVELMVVIAIIAILAAVAIPIYSSYKERAAIIESMNIIGNVKASIQNDINNNLDISQQTYDTPTGVTVTGSTSGATIDINLSQTSPQHFTNDNDIIRLSGVVVSGSTFQWTCSHNVNASTLTASNVPHTCSSTFSA

SEQ ID NO: 6 - protein, 135 aa, Francisella tularensis
MKKKMQKGFSLVELMVVIAIIAILAAVAIPMYSNYTTRAQLGSDLSALGGAKATVAERIANNNGDASQVTILQANAAANGLPSGASVAAGTISYPSTVSGATIQLAPTVSSGAITWTCNISGVSASQVPSNCNAI

SEQ ID NO: 7 - DNA, 33 bp, Artificial sequence
  Primer pilAcomp_1F
gcatgtcatatgaaaaagaaaatgcaaaaaggt

SEQ ID NO: 8 - DNA, 35 bp, Artificial sequence
  Primer pilAcomp_1R
gcatgtgaattcgattagatagcattacagttaga

SEQ ID NO: 9 - DNA, 21 bp, Artificial sequence
  Primer pilAcomp_4F
caacgccaaagattacatcac

SEQ ID NO: 10 - DNA, 21 bp, Artificial sequence
  Primer pilAcomp_4R
ttctgtcattcctgactcgac

SEQ ID NO: 11 - DNA, 61 bp, Artificial sequence
  Primer pilAflag_1R
gcatgtgaattcttatttatcatcatcatctttataatcgatagcattacagttagatggt

SEQ ID NO: 12 - DNA, 33 bp, Artificial sequence
  Primer pilA1-5prime
ccaaatctagacatgtactctaactacactaca

SEQ ID NO: 13 - DNA, 36 bp, Artificial sequence
  Primer pilA1-3prime
agattactcgagttattagatagcattacagttaga

SEQ ID NO: 14 - DNA, 21 bp, Artificial sequence
  Primer A
ttctcactagttgagttaatg

SEQ ID NO: 15 - DNA, 22 bp, Artificial sequence
  Primer B
ccattagctcaactagtgagaa

SEQ ID NO: 16 - DNA, 20 bp, Artificial sequence
  Primer C
ggggtagtactttaaatcct

SEQ ID NO: 17 - DNA, 19 bp, Artificial sequence
  Primer D
gcatcacacgagaggcatt

SEQ ID NO: 18 - DNA, 20 bp, Artificial sequence
  Primer E
gtgcctttgatcgtgcttac

SEQ ID NO: 19 - DNA, 20 bp, Artificial sequence
  Primer F
cttgagatgctgatacacct

SEQ ID NO: 20 - protein, 8 aa, Artificial sequence
  C-terminal Flag-epitope
DYKDDDDK

SEQ ID NO: 21 - protein, 35 aa, Francisella tularensis
MKKKMQKGFSLVELMVVIAIIAILAAVAIPMYSNY

SEQ ID NO: 22 - DNA, 120 bp, Francisella tularensis
aaggaaaacaaatcatgaaaaagaaaatgcaaaaaggtttctcactagttgagttaatggtagtaattgcaataattgcaatcttagcagctgtagcaataccgatttactcaaactaca

SEQ ID NO: 23 - DNA, 120 bp, Francisella tularensis
aaggaaaacaaatcatgaaaaagaaaatgcaaaaaggtttctcactagttgagttaatggtagtgatcgcgatcatcgctatcctagcagctgtagcgatcccgatgtactctaactaca

SEQ ID NO: 24 - DNA, 120 bp, Francisella tularensis
aagttatacaaattatgcaaacacacaaacaaacaggtttctcactagttgagctaatggtagtaattgcaataattgcaatcttagcagctgtagcaataccgatttactcaaactaca

SEQ ID NO: 25 - DNA, 120 bp, Francisella tularensis
aaggaaaacaaatcatgaaaaagaaaatgcaaacaggtttctcactagttgagctaatggtagtaattgcaataattgcaatcttagcagctgtagcaataccgatttactcaaactaca

SEQ ID NO: 26 - protein, 35 aa, Francisella tularensis
MQTHKQTGFSLVELMVVIAIIAILAAVAIPIYSNY